(19) United States
(12) Patent Application Publication
(10) Pub. No.: US 2017/0135715 A1
(43) Pub. Date: May 18, 2017

(54) BALLOON DISSECTION KIT WITH MULTIPLE BALLOONS

(71) Applicant: Covidien LP, Mansfield, MA (US)

(72) Inventors: Jay Breindel, Kensington, CT (US); Elias Hartoumbekis, New Haven, CT (US)

(21) Appl. No.: 15/341,021

(22) Filed: Nov. 2, 2016

Related U.S. Application Data
(60) Provisional application No. 62/254,391, filed on Nov. 12, 2015.

(51) Int. Cl. A61B 17/3209; A61B 17/34

(52) U.S. Cl. CPC A61B 17/3209; A61B 17/3423; A61B 17/3468; A61B 2017/320048

(57) ABSTRACT

The present disclosure is directed to a kit including a balloon dissector assembly to facilitate forming an anatomical space within the body of a patient during a surgical procedure. The dissector assembly includes an elongate shaft and a plurality of detachable balloons that are releasably connectable to the distal end of the elongate shaft. The balloons are of various shapes and sizes to permit a health care practitioner such as a surgeon to select a balloon having a configuration best suited for the surgical procedure being performed. The kit also includes spacers that may be releasably attached to an elongate shaft of the balloon dissector by a surgeon for limiting the depth of insertion of the elongate shaft of the dissector assembly into a body of a patient.

### CROSS-REFERENCE TO RELATED APPLICATIONS

This application claims the benefit of and priority to U.S. Provisional Patent Application No. 62/254,391 filed Nov. 12, 2015, the entire disclosure of which is incorporated by reference herein.

### TECHNICAL FIELD

The present disclosure relates to dissection devices for forming an anatomical space within a body, and, in particular, to balloon dissection devices, with multiple detachable balloons permitting selection of the optimal balloon for use with a patient.

### BACKGROUND

During certain surgical procedures, it is necessary to dissect tissue layers to form an anatomical space for accessing a surgical site, and within which surgical instruments may be manipulated. For example, in hernia repair surgery, it is necessary to form an anatomical operative cavity within the extra peritoneal space in order to dissect fascia tissue layers from the peritoneum and access the hernia site. Various balloon dissectors are known for performing the tissue dissection procedure used in hernia repair surgery. Depending on the patient and procedure being performed, different shapes and sizes of balloons are used in performing tissue dissection procedures.

While the currently known tissue dissection devices are useful, it would be beneficial to have a kit possessing multiple detachable balloons to optimize use with individual patients in surgical procedures requiring dissection of tissue layers.

### SUMMARY

The present disclosure is directed to a dissection and access kit including a balloon dissector assembly having a balloon dissector shaft and a plurality of detachable balloons. Each balloon is releasably attachable to the balloon dissector shaft prior to use and then disconnected following use in order to be disposed. By providing balloons that are releasably secured to the balloon dissector shaft, a clinician is able to select a balloon best suited to a particular surgical procedure.

In one aspect of the disclosure, a kit for a dissection and access system includes a cannula assembly having a cannula housing and an access cannula, an obturator insertable into the access cannula, and a dissector assembly insertable into the access cannula. The dissector assembly has an elongate shaft and a distal tip. At least one dissection balloon includes an inflatable portion and a connector for releasable attachment to the distal tip.

In embodiments, at least one spacer is releasably attached to the elongate shaft for limiting a depth of insertion of the elongate shaft of the dissector assembly into a body of a patient.

In some embodiments, a seal is provided for placement between the distal tip of the elongate shaft and the connector.

In certain embodiments, the distal tip of the elongate shaft and the connector possess threads permitting the distal tip and connector to be threadably attached.

In embodiments, the distal tip of the elongate shaft is releasably attached to the connector by a bayonet configuration.

In some embodiments, the distal tip possesses an open end with slots on an inner surface of the elongate shaft. The slots extend proximally along the inner wall of the elongate shaft to form recesses thereon. The connector possesses a proximal end configured to fit within the open end of elongate shaft. An outer surface of the proximal end possesses tabs that extend from the outer surface of the connector and are configured to be slidably received within the slots of the open end of the elongate shaft.

In certain embodiments, the distal tip of the elongate shaft is releasably attached to the connector by a locking collar.

In embodiments, the at least one dissection balloon includes a plurality of round balloons of different sizes.

In some embodiments, the at least one dissection balloon includes a plurality of oval balloons of different sizes.

In certain embodiments, the at least one dissection balloon includes at least one round balloon and at least one oval balloon.

In embodiments, the at least one spacer has a shape of a disc having a lateral slit therein and a hole at its center.

In another aspect of the disclosure, a method of forming a balloon dissector assembly for use in a system for dissecting tissue is disclosed including the steps of obtaining a kit including a balloon dissector assembly having an elongate shaft and a distal tip, and a plurality of dissection balloons, wherein each of the dissection balloons has an inflatable portion and a connector for releasable attachment to the distal tip; selecting one of the plurality of dissection balloons for use; and releasably attaching the selected dissection balloon to the distal tip of the elongate shaft.

In embodiments, the kit further includes at least one spacer for limiting a depth of insertion of the elongate shaft of the balloon dissector assembly into a body of a patient.

In some embodiments, the method further includes placing the at least one spacer along the elongate shaft of the balloon dissector assembly to limit a depth of insertion of the elongate shaft of the balloon dissector assembly into a body of a patient.

In embodiments, the method further includes placing a seal between the distal tip of the elongate shaft and the connector.

In embodiments, the distal tip of the elongate shaft and the connector are threadably attached.

In embodiments, the distal tip of the elongate shaft is releasably attached to the connector by a bayonet configuration.

In embodiments, the distal tip of the elongate shaft is releasably attached to the connector by a locking collar.

In embodiments, a round balloon of desired size is selected as the dissection balloon.

In embodiments, an oval balloon of desired size is selected as the dissection balloon.

In embodiments, the at least one spacer has a shape of a disc having a lateral slit therein and a hole at its center. The lateral slit is placed along the elongate shaft of the dissector assembly and the spacer is pushed perpendicular to the axis of the elongate shaft so that the sides of the slit move away from the shaft until the shaft lies within the hole in the center of the spacer.

### BRIEF DESCRIPTION OF THE DRAWINGS

Various embodiments are described herein with reference to the drawings wherein:

FIG. 1 is a perspective view of a balloon dissector capable of attachment to multiple balloons in accordance with an embodiment of the present disclosure;

FIG. 2 is a perspective view of the balloon dissector shown in FIG. 1 having a single inflated balloon attached thereto in accordance with the present disclosure;

FIG. 3 includes cross-sectional views of various round balloons that may be provided with a kit in accordance with the present disclosure;

FIG. 4 includes cross-sectional views of various oval balloons that may be provided with a kit in accordance with the present disclosure;

FIG. 5 is a cross-sectional view of the distal tip of the balloon dissector shown in FIG. 1 threadably attached to a balloon in accordance with the present disclosure;

FIG. 6 is an enlarged cross-sectional view of the indicated area of detail shown in FIG. 5;

FIG. 7 is a cross-sectional view showing details of the threads for the balloon dissector and balloon shown in FIG. 6 in accordance with the present disclosure;

FIG. 8 is a perspective view of the balloon dissector and balloon for an alternate embodiment of the present disclosure, where the balloon dissector and balloon are connected by a bayonet attachment method;

FIG. 9 is an enlarged view of the coupling structure of the balloon dissector and balloon in accordance with the embodiment of FIG. 8;

FIG. 10 is an enlarged view of the coupling structure of the balloon dissector and balloon for assembly in accordance with the embodiment of FIG. 8;

FIG. 11 is an enlarged view showing steps for attaching the balloon dissector and balloon in accordance with the embodiment of FIG. 8;

FIG. 12 is an enlarged view showing the balloon dissector attached to the balloon after assembly in accordance with the embodiment of FIGS. 8-11;

FIG. 13 is a perspective view of the balloon dissector and balloon attached using a locking collar in accordance with the present disclosure;

FIG. 14 is an enlarged view of the assembly of the balloon dissector and balloon in accordance with the embodiment of FIG. 13;

FIG. 15 is an enlarged view showing attachment of the balloon dissector and balloon in accordance with the embodiment of FIGS. 13-14;

FIG. 16 is an enlarged view showing attachment of the balloon dissector and balloon in accordance with the embodiment of FIGS. 13-16;

FIG. 17 is a perspective view of a spacer to be used with a kit of the present disclosure;

FIG. 18 is a perspective view of a variable length system formed of a kit of the present disclosure;

FIG. 19 is a perspective view of a variable length system formed of a kit of the present disclosure using spacers to shorten the working length of the system;

FIG. 20 is a perspective view of a variable length system of the present disclosure without spacers; and

FIG. 21 is an exploded view of the components of a variable length system formed of a kit of the present disclosure.

### DETAILED DESCRIPTION

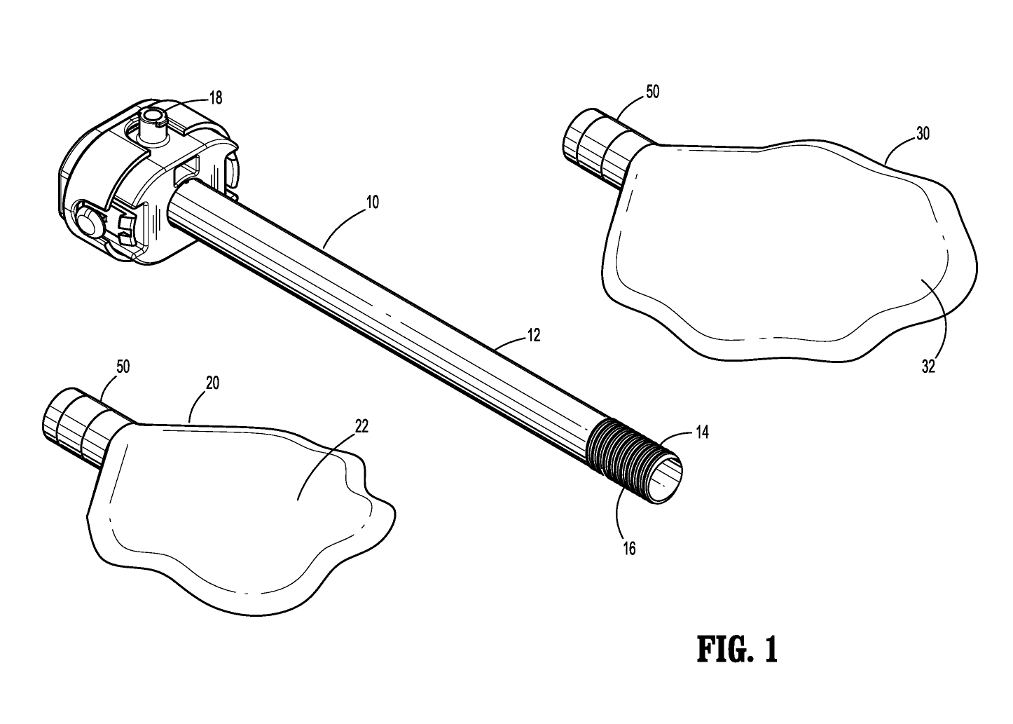

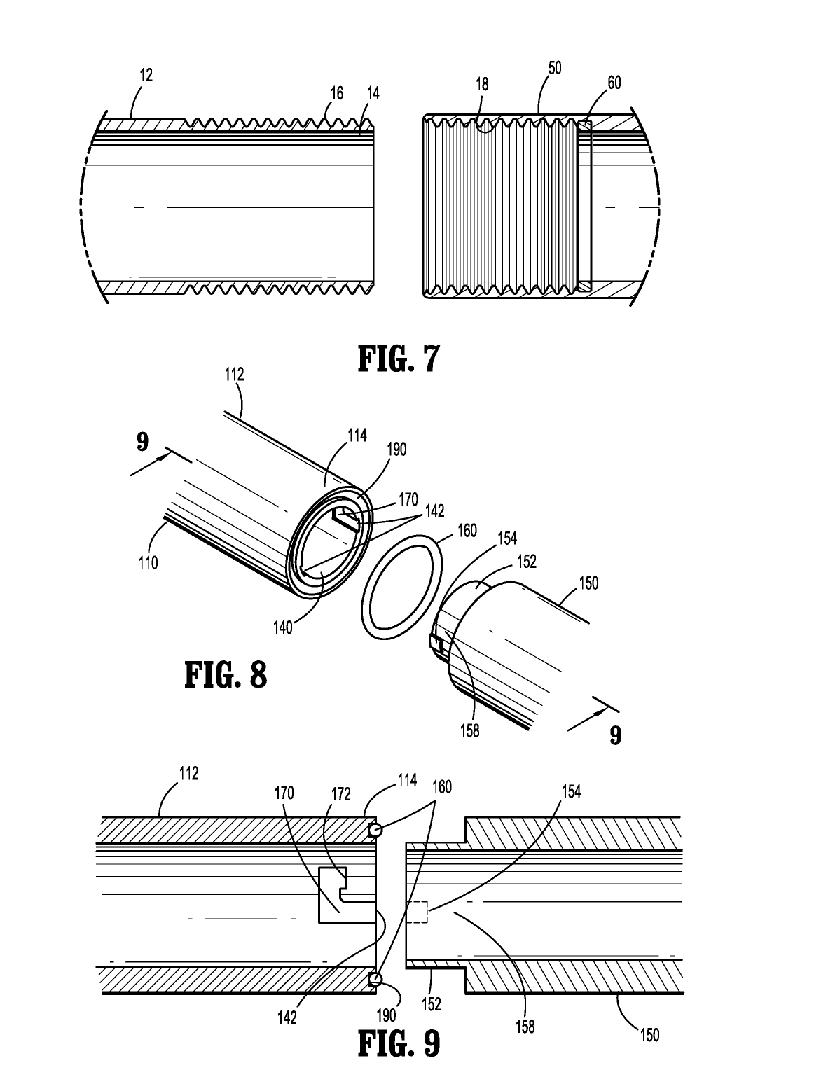

Dissection and access kits including a balloon dissector assembly in accordance with embodiments of the present disclosure are shown in FIGS. 1-21. The kits include a balloon dissector assembly including an elongated shaft and a plurality of detachable balloons connectable to the elongate shaft. The provision of detachable balloons allows a surgeon to select a balloon having a configuration best suited for a particular surgical procedure as discussed in detail below.

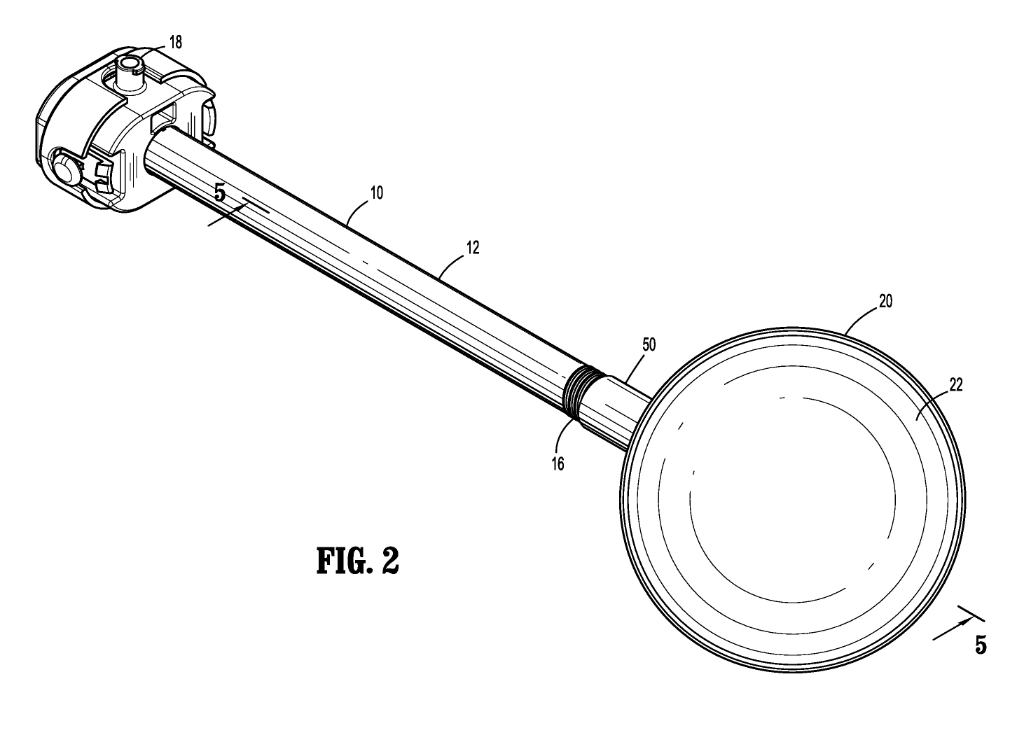

Referring to FIGS. 1 and 2, the kit of the present disclosure includes a balloon dissector assembly 10 having an elongate shaft 12 and a distal tip 14, with varying balloons 20 and 30, having different shapes and sizes, for attachment to distal tip 14 of the elongate shaft 12. Balloons 20 and 30 include both an inflatable portion 22, 32, and a connector 50, 50a, for attachment with the distal tip 14 of the elongate shaft 12 of the balloon dissector assembly 10. As depicted in FIG. 1, in embodiments the distal tip 14 may have threads 16 suitable for engaging threads (not shown) within the connector 50, 50a by threadably attaching together the distal tip 14 and the connector 50, 50a.

The elongate shaft 12 includes an inflation port 18 for inflating the dissection balloon 20, 30. In order to inflate the dissection balloon 20, 30 a source of inflation pressure is releasably attached to the inflation port 18 and pressurized fluid is introduced through the inflation port 18 and communicated along axis 5-5 to the dissection balloon 20, 30. FIG. 2 shows the balloon dissector assembly 10 with the balloon 20 attached thereto and inflated.

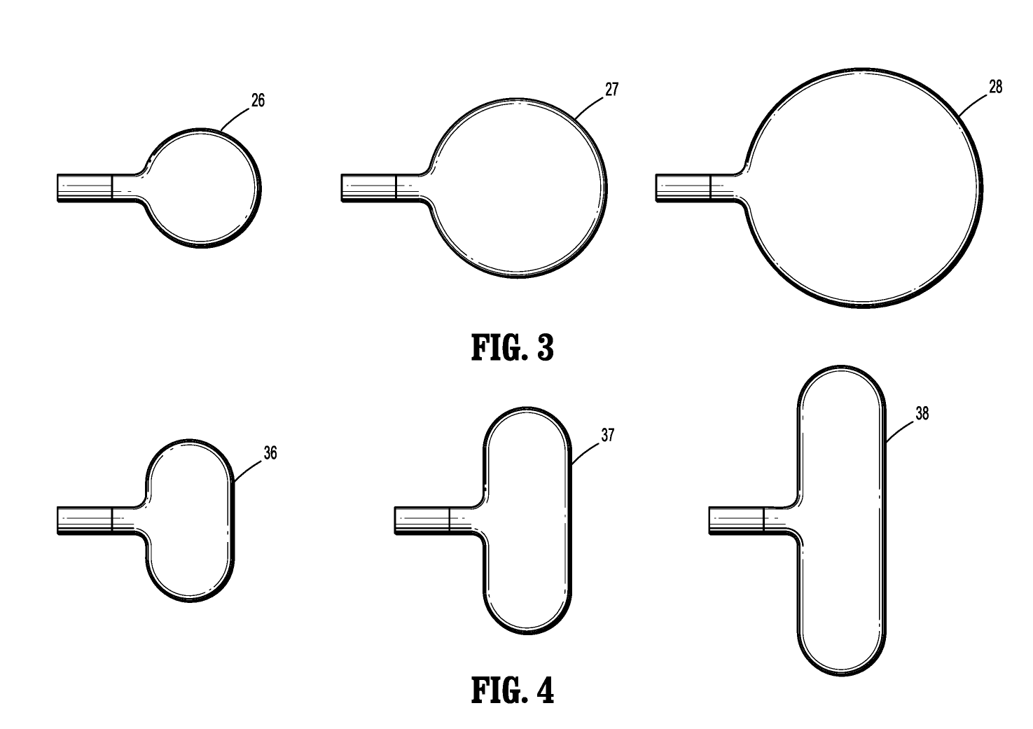

As noted above, balloons of varying shapes and sizes may be supplied with a kit of the present disclosure permitting increased variability in the procedures that require dissector balloons of varying shapes. Suitable shapes include round balloons as depicted in FIG. 3 as round balloons 26, 27, and 28, and oval balloons as depicted in FIG. 4 as oval balloons 36, 37, and 38. The balloons may be elastic, inelastic or a combination of materials having both characteristics. The selection of balloon is left up to the surgeon. In some cases, a round balloon may be made of an elastic material and a laterally extending oval balloon may be made of an inelastic material.

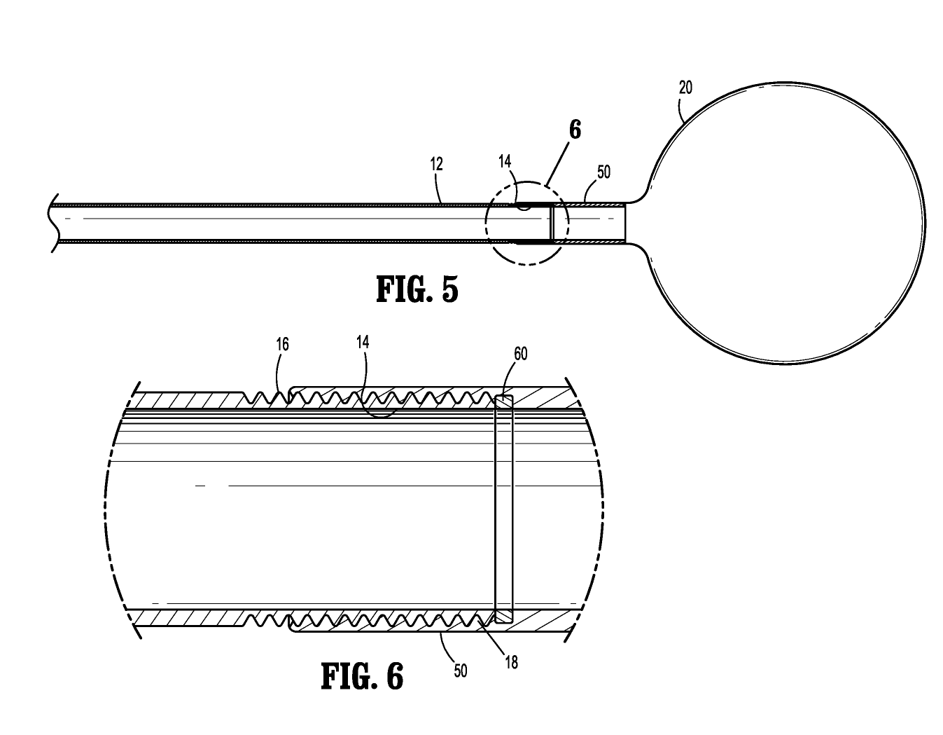

Turning to FIGS. 5-7, greater detail of the attachment of the connector 50 of the balloon 20 for attachment with the distal tip 14 of the elongate shaft 12 of the balloon dissector assembly 10 is shown. As depicted in FIGS. 5, 6 and 7, the distal tip 14 of the elongate shaft 12 is of a male configuration having a smaller circumference than the circumference of the connector 50, which has a female configuration. The distal tip 14 has threads 16 thereon suitable for engaging threads 18 within the connector 50 for threadably attaching the distal tip 14 and the connector 50. The connector 50 of the balloon 20 also possesses a seal 60 disposed therein, which permits attachment of the balloon 20 to the distal tip 14 in a secure and air-tight manner.

While FIGS. 5-7 depict the distal tip 14 of the elongate shaft 12 having a male configuration and the connector 50 having a female configuration, in embodiments (not shown) reverse configurations are contemplated, i.e., the distal tip 14 having a female configuration and the connector 50 having a male configuration.

Other coupling configurations for attaching a distal tip 114 of an elongate shaft 112 of a balloon dissector assembly 110 with a connector 150 of balloons (not shown) are contemplated. For example, as depicted in FIGS. 8-12, a bayonet configuration for attaching the distal tip 114 with the connector 150 is provided. As depicted in FIG. 8, the distal tip 114 of the elongate shaft 112 of the balloon dissector assembly 110 possesses an open end 140 with slots 142 on the inner surface of the elongate shaft 112. As depicted in FIGS. 8 and 9, the slots 142 extend proximally along an inner wall of the elongate shaft 112, thereby forming grooves 170 thereon. (A second slot 142 forms a second corresponding groove, 170, not shown in FIG. 8 or 9, but shown in FIG. 11.) The connector 150 of the balloon 120 possesses a proximal end 152 having a male configuration which is received within the open end 140, having a female configuration, of the elongate shaft 112. In addition, an outer surface 158 of the proximal end 152 of the connector 120 has tabs 154 (second tab 154 is not shown) extending from the outer surface 158 of the connector 120. The tabs 154 are slidably received within the slots 142 of the open end 140 of the elongate shaft 112. As shown in FIG. 8, the open end 140 of the elongate shaft 112 may also possess a recess 190 therein for placement of a seal 160, e.g., an o-ring, which may be placed between the elongate shaft 112 and the connector 120.

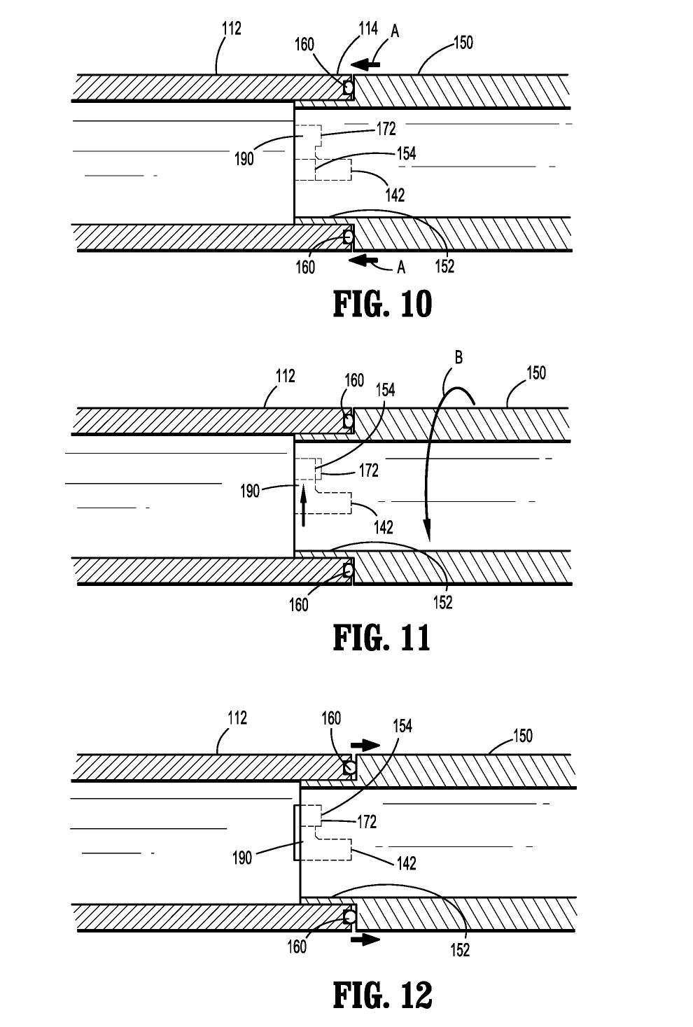

Turning to FIGS. 9-12, in use, the seal 160 is placed in the recess 190 in the open end 140 of the elongate shaft 112 of the balloon dissector assembly 110 (FIG. 9). The proximal end 152 of the connector 150 is then inserted into the open end 140 of the elongate shaft 112 in the direction indicated by arrows “A” in FIG. 10 so that the tabs 154 extending from the outer surface 158 of the connector 150 are slidably received in the slots 142 of the open end 140 of the elongate shaft 112. The connector 150 and the distal tip 114 of the elongate shaft 112 are then pressed together, with the seal 160 compressed and acting like a spring, so that the tabs 154 travel proximally from the slots 142 to the grooves 170 along axis 9-9. As shown in FIG. 11, as the grooves 170 possess an “L” shape, once the tabs 154 can travel no further in the proximal direction along the axis 9-9, the connector 120 is rotated around the axis 9-9 in the direction indicated by arrow “B” in FIG. 11 so that the tabs 154 travel along the grooves 170 in a direction perpendicular to the axis 9-9. As shown in FIG. 12, upon release of the connector 150 and the distal tip 114 of the elongate shaft 112, the seal 160, again acting like a spring, is decompressed, and permits the tabs 154 to move distally along the axis 9-9 into the recesses 172 (only 1 recess is depicted) at the end of the grooves 170 which lock the tabs 154 into the grooves 170, preventing further twisting or movement of the connector 120 relative to the elongate shaft 112 (in the absence of additional compressive/twisting forces).

While FIGS. 8-12 depict the distal tip 114 having certain structure and the connector 150 having corresponding structures to permit attachment of the distal tip 114 to the connector 150 by a bayonet attachment configuration, in embodiments (not shown) reverse configurations are contemplated, i.e., the distal tip 114 possesses the structures shown for the connector 150 and the connector 150 possesses the structures for the distal tip 114.

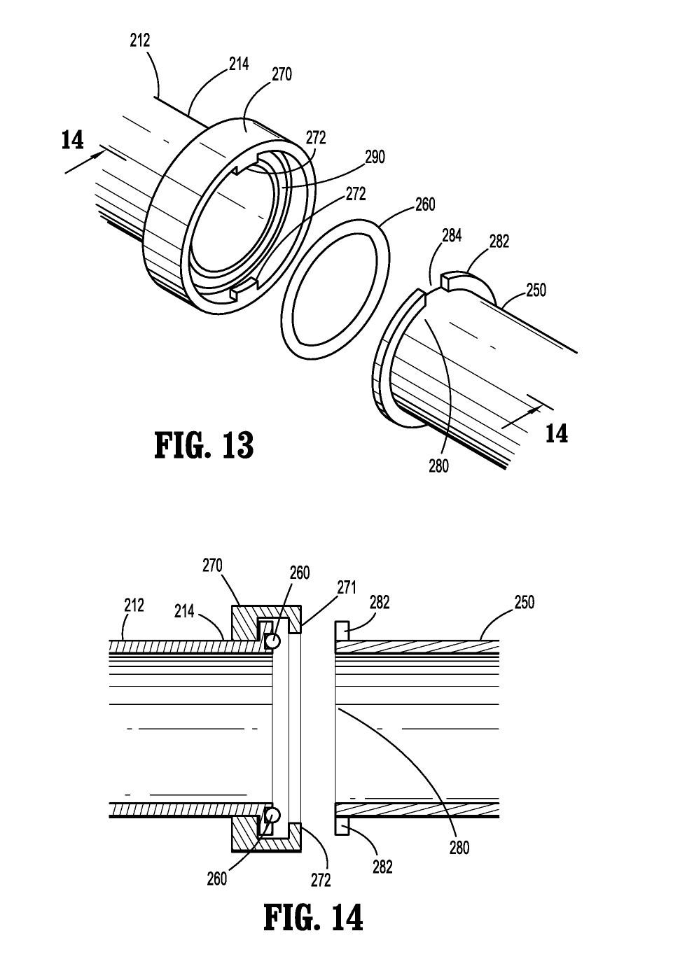

Yet another configuration for attaching a distal tip 214 of an elongate shaft 212 of a balloon dissector assembly with a connector 250 of a balloon is depicted in FIGS. 13-16, which show a locking collar configuration for attaching the distal tip 214 of the elongate shaft 212 with the connector 250. As depicted in FIG. 13, the distal tip 214 of the elongate shaft 212 of a balloon dissector assembly possesses a locking collar 270 thereon with protrusions 272 on the inner surface of the locking collar 270. As depicted in FIGS. 13 and 14, the connector 250 possesses a proximal end 280 with an incomplete ring 282 thereon having gaps 284, (second gap 284 is not shown in FIG. 13), which are configured to allow the protrusions 272 of the locking collar 270 to pass distally beyond the ring 282. As shown in FIG. 13, the elongate shaft 212 may also possess a recess 290 therein for placement of a seal 260, which may be placed between the elongate shaft 212 and the connector 250 of the balloon.

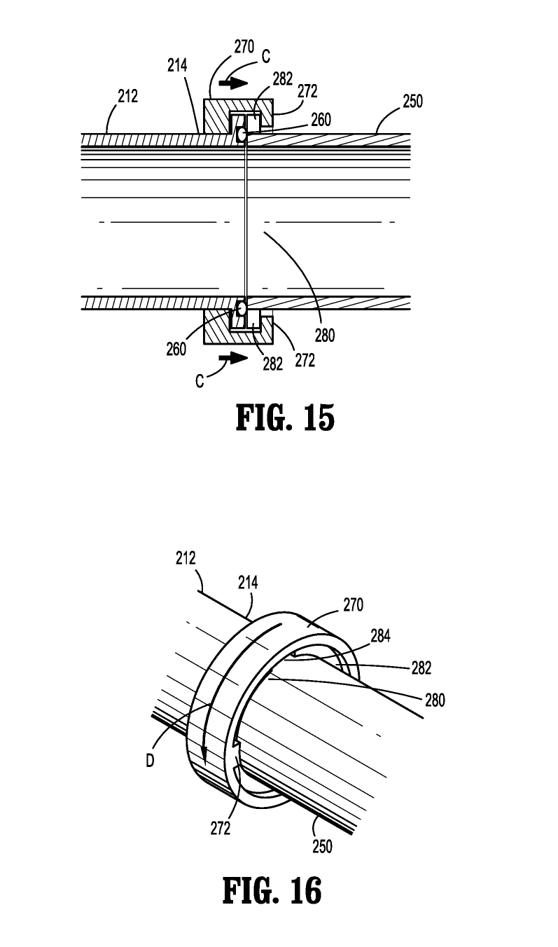

Turning to FIGS. 15 and 16, in use, the seal 260 is placed in the recess 290 of the distal tip 214 of the elongate shaft 212 of the balloon dissector assembly. The locking collar 270 is then placed over the proximal end 280 of the balloon connector 250 in the direction indicated by arrow “C” in FIG. 15 so that the protrusions 272 on the inner surface of the locking collar 270 can pass through the gaps 284 of the incomplete ring 282. The connector 250 and the distal tip 214 of the elongate shaft 212 are then pressed together, with the seal 260 compressed and acting like a spring, so that the protrusions 272 pass through the gaps 284, allowing the distal end of the locking collar 270 to pass distally beyond the ring 282. The locking collar 270 is then rotated around axis 14-14 in a direction indicated by arrow “D” in FIG. 16, so that the protrusions 272 on the inner surface of the locking collar 270 abut the incomplete ring 282. Upon release of the locking collar 270, the seal 260, again acting like a spring, is decompressed, and the protrusions 272 frictionally engage with the incomplete ring 282, preventing further twisting or movement of the locking collar 270 relative to the balloon connector 250 (in the absence of additional compressive/twisting forces).

While FIGS. 13-16 depict the distal tip 214 of the elongate shaft 212 having certain structure and the connector 250 having corresponding structures to permit attachment of the distal tip 214 to the connector 250 by the locking collar 270, in embodiments (not shown) reverse configurations are contemplated, i.e., the distal tip 214 possesses the structures shown for the connector 250 and the connector 250 possesses the structures for the distal tip 214.

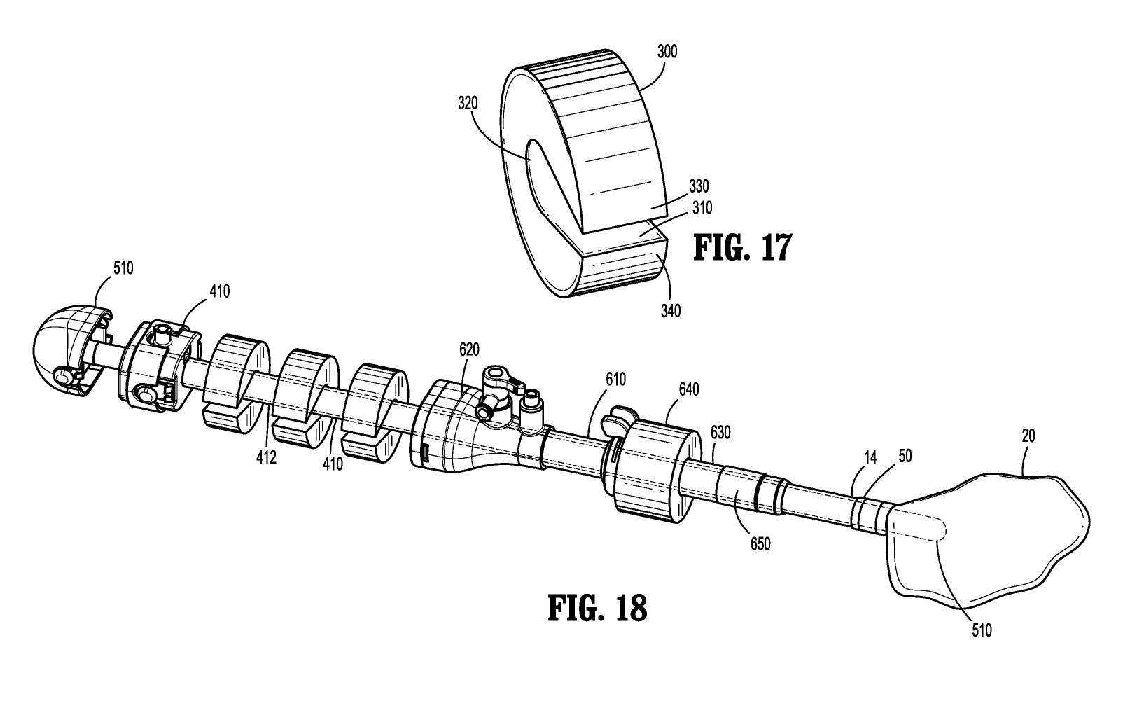

Referring to FIGS. 17 and 18, the balloon dissector assembly 410 of the present disclosure may be utilized with an obturator 510 and a cannula assembly 610 for use in endoscopic and laparoscopic minimally invasive procedures. An access cannula 630 of the cannula assembly 610 remains in place for use during the minimally invasive procedure, and the obturator 510 includes a sharp tip for penetrating the body cavity.

In embodiments, to further enhance the variability of the kit of the present disclosure, removable spacers 300 may be provided to vary the working length of the balloon dissector assembly 410. More specifically, the removable spacers 300 may be added between the dissector assembly 410 and the cannula assembly 610 to reduce the overall working length of the shaft 12 of the balloon dissector assembly 410, i.e., to reduce the extent to which the balloon dissector assembly 10 extends from the distal end of cannula assembly 610 into a body cavity. Suitable spacers 300 are depicted in FIG. 17. Each spacer 300 is in the shape of a disc, having a lateral slit 310 therein and a hole 320 in the center of the spacer 300. The slit 310 permits sliding of the spacer 300 onto the elongate shaft 412 of the balloon dissector assembly 410 in such a way that when the slit 310 is placed adjacent the shaft 412 and the spacer 300 is pushed perpendicular to the axis of the elongate shaft 412, the sides 330, 340 of the slit 310 are moved away from the shaft 412 until the shaft 412 comes to lie within the hole 320 in the center of the spacer 300, at which point the sides 330, 340 of the slit 310 return to their original configuration and the spacer 300 encompasses the shaft 412.

The provision of a spacer between the cannula assembly 610 and the balloon dissector assembly 410 makes it possible to vary the depth of insertion of a balloon 20 of the balloon dissector assembly 410 of the present disclosure into a body cavity. Placement of more than one spacer 300 may further limit the depth of insertion of the balloon dissector assembly 410, thus providing a practitioner with a means to vary depth of insertion to suit the needs of a patient in view of the procedure being performed. As the spacer(s) 300 are releasably mounted on the instrument, the spacer 300 can be fitted on the medical instrument when the need arises. Moreover, the releasability of the spacer 300 permits altering the depths of insertion of the balloons 20, 30 of the balloon dissector assembly 410. This can be done so that the use of the balloon dissector assembly 410 can be adapted with the greatest possible variation to the anatomical circumstances in question.

FIG. 18 depicts a system of the present disclosure showing the balloon dissector assembly 410, the dissector obturator 510, and the balloon cannula assembly 610. Spacers 300, 320, and 330 are shown as they would be placed on the outer surface of the elongate shaft 412 of the balloon dissector assembly 410, to adjust the working length of the system. The cannula assembly 610 includes a cannula housing 620 and the access cannula 630 with a skin seal 640 and an anchoring balloon 650 thereon. The dissector obturator assembly 510 and the balloon dissector assembly 410 are insertable into the access cannula 630.

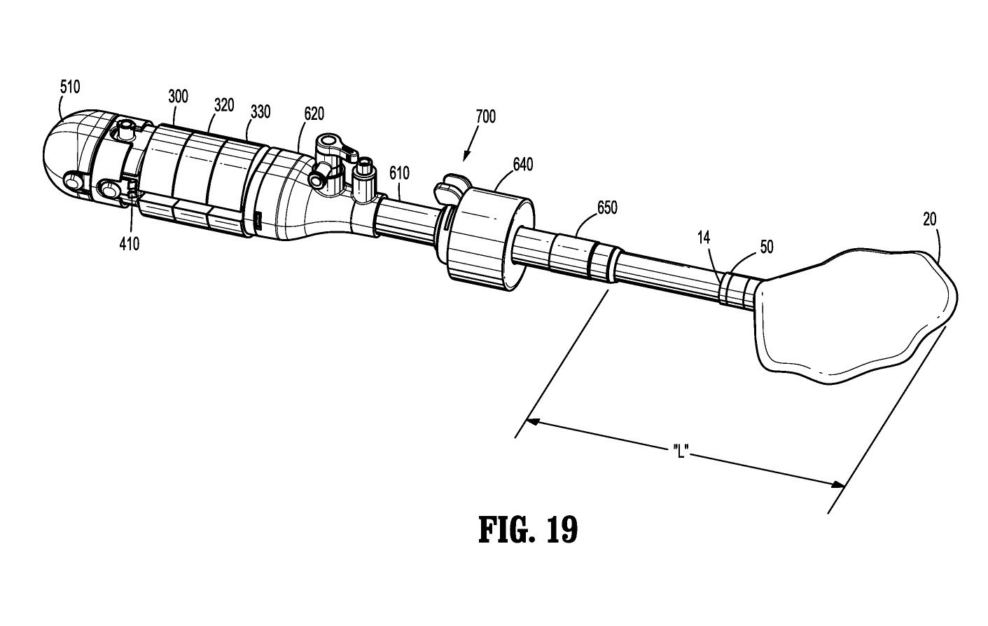

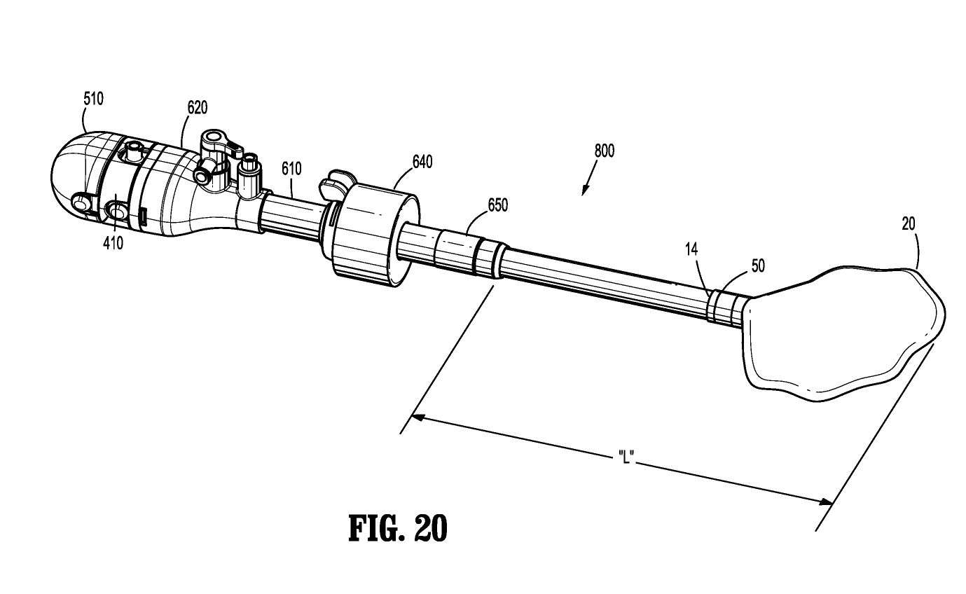

FIG. 19 is a depiction of a variable length system 700, showing placement of the spacers 300, 320, and 330 on the outer surface of the elongate shaft 412 of the balloon dissector assembly 410, giving a defined working length “L” for the system. FIG. 20 shows a similar variable length system 800, lacking any spacers on the outer surface of the elongate balloon dissector assembly 410, giving a larger defined working length “L′” for the system compared with FIG. 19.

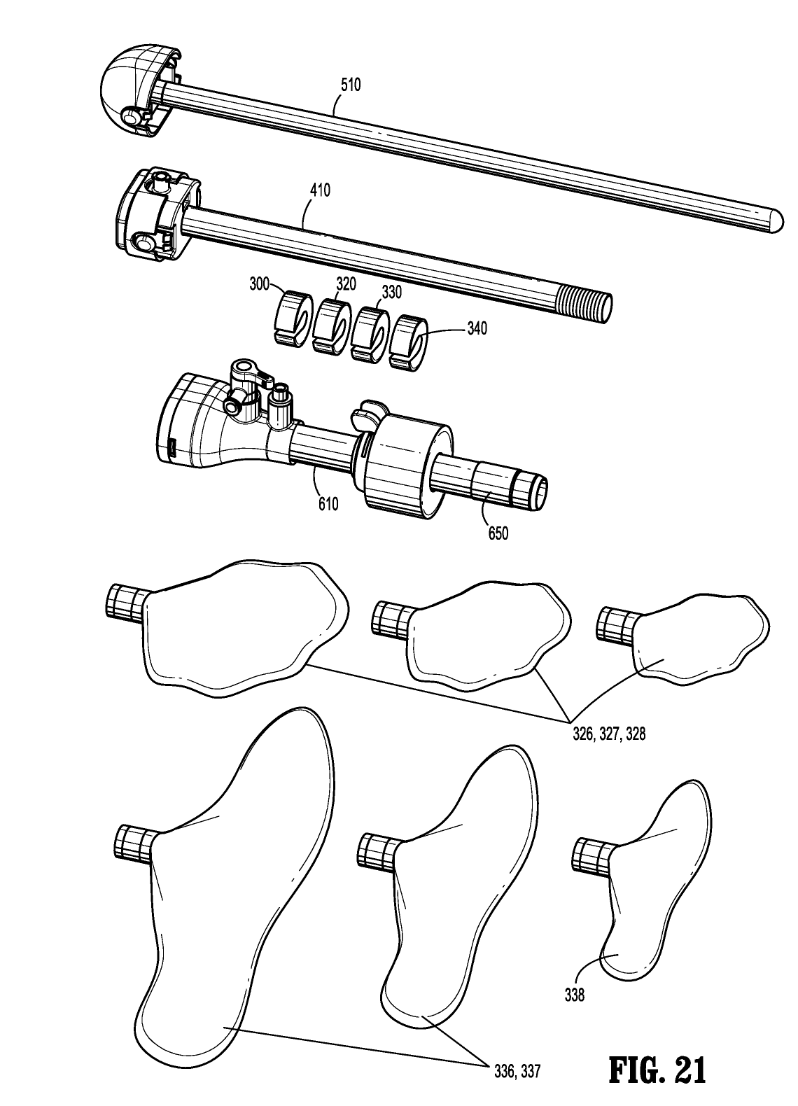

FIG. 21 is a depiction of the unassembled variable length system of the present disclosure, showing the balloon dissector assembly 410, separate from the dissector obturator 510, and the balloon cannula assembly 610. The spacers 300, 320, 330, and 340 are shown separately, as are the round balloons 326, 327, and 328, and the oval balloons 336, 337, and 338.

Additional details regarding the system, including the components utilized in the system, methods for its production, and methods for its use, include those disclosed in U.S. Pat. No. 8,540,745, the entire disclosure of which is incorporated by reference herein.

The balloon dissector assembly 410 is used for dissecting tissue along natural tissue planes in general, laparoscopic, vascular endoscopic, plastic or reconstructive surgery or other procedures requiring the separation of tissue. Where prior devices would require different working lengths and different balloons thereon, requiring multiple systems on hand for use in a procedure, kits of the present disclosure permit customization of both the balloon utilized for dissection as well as the working length of the system.

The material of the balloon may be elastic, so as to follow a path of least resistance in the body, inelastic so as to assume a predetermined shape upon inflation, or a combination of elastic and inelastic materials. The balloon dissector and cannula assembly may be used in hernia repair, bladder neck suspension or other procedures requiring the separation of tissue. The balloon dissector and cannula assemblies can be made from any medical grade material, including metals and plastics. The apparatus is made using well-known techniques.

In embodiments, round balloons may be formed of an elastic material, while laterally extending oval balloons may be inelastic.

The dissection balloon may be inflated with any medical grade fluid, such as saline, CO₂, or any other fluid. The balloons may be inflated using a syringe, mechanically or manually operated pump or other means. The ports for inflating the balloons may be used with one-way valves, check valves, or any other valve arrangement for inflating the balloons.

The shape of the dissection balloon can vary upon the area of use in the anatomical structure. For example, the balloon may have the round shape of a globe, a flattened round shape, may include a longitudinally oval shape or other shapes such as kidney shaped, laterally extending, etc., depending on the need of the surgeon. The selection of balloon is left up to the surgeon. Once the desired balloon is selected by the surgeon, it is affixed to the distal tip of the elongate shaft of the balloon dissector assembly as described in the above embodiments, which include by use of threadable attachment, the use of a bayonet attachment, the use of a locking collar, etc.

Similarly, as noted above, the desired working length of the system may be optimized for the patient in view of the intended procedure with the use of the spacers affixed to the elongate shaft of the balloon dissector assembly, and/or any other shaft of the dissector obturator or balloon cannula assembly. Again, the more spacers added to the shaft, the shorter the working length of the system, which is decided upon by the surgeon at the time of the procedure.

Once the kit of the present disclosure has been used to assemble a desired system, a suitably sized incision is made in the patient's skin. Next, the assembled balloon dissector assembly 410 and the balloon cannula assembly 610 are inserted into the incision, using the dissector obturator 510 to tunnel a passage beyond the point of incision.

Inflation pressure is supplied through the inflation port 28 from a suitable outside source and is communicated to the dissection balloon 20. As pressure is applied, the dissection balloon 20 expands. The expansion of dissection balloon dissects surrounding tissue along natural tissue planes. Once the desired space is created, the dissection balloon 20 is deflated by disengaging the inflation source from the inflation port 28 and/or connecting the inflation port 20 to a source of vacuum.

The balloon dissector assembly 410 is removed from the cannula assembly 610 and surgical instruments are introduced to the surgical site through the cannula. Examples of such surgical instruments include, but are not limited to, endoscopes, surgical suturing devices, and surgical device applicators.

For example, in alternative embodiments, the dissector obturator 510 is removed and replaced with an endoscope. Then, the balloon dissector assembly 410 and the cannula assembly 610 are inserted into the skin incision and the dissector balloon 20 is inflated as discussed above. The scope is used for supporting the balloon 20, as well as for viewing the space to be dissected prior to, during, and after dissection.

It will be understood that various modifications may be made to the embodiments disclosed herein. For example, other configurations of securing a balloon to a balloon dissector assembly may be provided to form a system of the present disclosure. Additionally, other balloon shapes and construction such as, for example elastic, inelastic, oval, kidney shaped, along with constructions providing differential expansion characteristics may be provided. Further, the terminology of similar components with the various embodiments should not be construed as specific to any particular embodiment. Thus, the above description should not be construed as limiting, but merely as exemplifications of preferred embodiments. Those skilled in the art will envision other modifications within the scope and spirit of the claims appended hereto.

What is claimed is:

1. A kit for a dissection and access system comprising:
a cannula assembly having a cannula housing and an access cannula;
an obturator insertable into the access cannula;
a dissector assembly insertable into the access cannula, the dissector assembly having an elongate shaft having a distal tip; and
at least one dissection balloon including an inflatable portion and a connector for releasable attachment to the distal tip of the elongate shaft.

2. The kit of claim 1, further comprising at least one spacer releasably attachable to the elongate shaft for limiting a depth of insertion of the elongate shaft of the dissector assembly into a body of a patient.

3. The kit of claim 1, further comprising a seal for placement between the distal tip of the elongate shaft and the connector.

4. The kit of claim 1, wherein the distal tip of the elongate shaft and the connector possess threads permitting the distal tip and connector to be threadably attached.

5. The kit of claim 1, wherein the distal tip of the elongate shaft and the connector include structure permitting the distal tip to be releasably attachable to the connector by a bayonet configuration.

6. The kit of claim 5, wherein the distal tip possesses an open end with slots on an inner surface of the elongate shaft, the slots extending proximally along the inner wall of the elongate shaft to form recesses thereon, the connector having a proximal end configured to fit within the open end of elongate shaft, an outer surface of the proximal end having tabs extending from the outer surface of the connector, the tabs being configured to be slidably received within the slots of the open end of the elongate shaft.

7. The kit of claim 1, further comprising a locking collar for releasably attaching the distal tip of the elongate shaft to the connector.

8. The kit of claim 1, wherein the at least one dissection balloon includes a plurality of round balloons of different sizes.

9. The kit of claim 1, wherein the at least one dissection balloon includes a plurality of oval balloons of different sizes.

10. The kit of claim 1, wherein the at least one dissection balloon includes at least one round balloon and at least one oval balloon.

11. The kit of claim 1, wherein the at least one spacer has a shape of a disc, the disc having a lateral slit therein and a hole at its center.

12. A method of forming a balloon dissector assembly for use in a system for dissecting tissue comprising:
obtaining a kit including a balloon dissector assembly having an elongate shaft and a distal tip, and a plurality of dissection balloons, each of the dissection balloons having an inflatable portion and a connector for releasable attachment to the distal tip;
selecting one of the plurality of dissection balloons for use; and
releasably attaching the selected dissection balloon to the distal tip of the elongate shaft.

13. The method of claim 12, wherein the kit further includes at least one spacer for limiting a depth of insertion of the elongate shaft of the balloon dissector assembly into a body of a patient and the method further includes placing the at least one spacer along the elongate shaft of the balloon dissector assembly to limit a depth of insertion of the elongate shaft of the balloon dissector assembly into a body of a patient.

14. The method of claim 12, further comprising placing a seal between the distal tip of the elongate shaft and the connector.

15. The method of claim 12, wherein the distal tip of the elongate shaft is releasably attached to the connector by threads.

16. The method of claim 12, wherein the distal tip of the elongate shaft is releasably attached to the connector by a bayonet configuration.

17. The method of claim 12, wherein the distal tip of the elongate shaft is releasably attached to the connector by a locking collar.

18. The method of claim 12, wherein a round balloon of desired size is selected as the dissection balloon.

19. The method of claim 12, wherein an oval balloon of desired size is selected as the dissection balloon.

20. The method of claim 12, wherein the at least one spacer has a shape of a disc, the disc having a lateral slit therein and a hole at its center, and the method further includes placing the lateral slit along the elongate shaft of the dissector assembly and pushing the spacer perpendicular to the axis of the elongate shaft so that the sides of the slit move away from the shaft until the shaft lies within the hole in the center of the spacer.
